(19) United States
(12) Patent Application Publication
(10) Pub. No.: US 2018/0125117 A1
(43) Pub. Date: May 10, 2018

(54) Enhanced Modular Electronic Cigarette Assembly with Disposable Elements Including Tanks

(71) Applicant: VOODOO SCIENCE LLC, Los Angeles, CA (US)

(72) Inventors: Kevin DeMeritt, Los Angeles, CA (US); Nicholas Grayson Sullivan, La Canada/Flintridge, CA (US); Jeffrey Charles Raber, Pasadena, CA (US); Steve Piorek, Los Angeles, CA (US)

(21) Appl. No.: 15/541,359

(22) PCT Filed: Dec. 30, 2015
(86) PCT No.: PCT/US2015/068113

§ 371 Date: Jun. 30, 2017

Related U.S. Application Data
(63) Continuation of application No. 14/333,920, filed on Jul. 17, 2014.
Application No. 14/333,920 is a continuation-in-part of application No. 14/203,560, filed on Mar. 11, 2014.
(60) Provisional application No. 62/098,861, filed on Dec. 31, 2014.

(51) Int. Cl. A24F 47/00; A61M 15/00; A61M 15/06

(52) U.S. Cl. CPC A24F 47/008; A61M 15/002; A61M 15/06; A61M 15/0086

(57) ABSTRACT

Radically improved modular devices, system and methods make it more convenient for a user to exchange the flavoring or physiological active species with ease and without mixing of previously loaded mixtures. Likewise devices, systems, and methods for introducing flavoring, nicotine, or other chemical species desired for consumption by the user into e-cigarette vapor without heating them, thus avoiding excessive pyrolysis or degradation to those ingredients is taught. Safer alternatives to current e-liquid containers and devices, making them less accessible to children, and making harmful exposure to liquid nicotine less likely in general are shown.

### FIELD OF THE INVENTION

The present invention relates generally to breathable fluid delivery devices such as electronic cigarettes, and more specifically to configurations including interchangeable and disposable parts for introduction of chemical species into a breathable fluid stream provided thereby.

### BACKGROUND OF THE INVENTION

Smoking electronic cigarettes, or “vaping,” is becoming an increasingly popular hobby. Some users smoke them in addition to as an alternative to traditional cigarettes and other tobacco products. Others use them as an aid in quitting smoking. The current electronic cigarette trend began in China when pharmacist Hon Lik developed a device for aerosolizing liquid nicotine as a way to help him quit smoking. See Demick, “A high-tech approach to getting a nicotine fix,” Los Angeles Times, Apr. 25, 2009. However, similar devices were developed earlier, such as Herbert A. Gilbert's smokeless non-tobacco cigarette, described in U.S. Pat. No. 3,200,819, incorporated herein by reference.

Today's electronic cigarettes (also known as e-cigarettes, e-cigs, and personal vaporizers) are battery-powered vaporizing devices, which produce an inhalable aerosol (referred to as “vapor”) by atomizing a liquid solution (“e-liquid” or “e-juice”), generally using a heating element such as a metal coil. E-liquid is typically composed of a mixture of one or more of propylene glycol (pg) and vegetable glycerin (vg), combined with flavorings, nicotine, or other psychoactive chemical ingredients.

Some vaping devices include a tank, which may hold for example 1 mL, 2 mL, or 5 mL of e-liquid at a time. A wick draws the e-liquid into an atomizer, which heats it with a coil and produces vapor. A 2 mL tank may allow a user to take about 100 puffs or drags—about the equivalent of 10 cigarettes—before having to refill the tank. Tanks can generally be filled by removing the mouthpiece and squeezing more e-liquid into the tank from a dropper.

Other vaping devices, known as “drippers” require the user to manually add a few drops of e-liquid directly to the coils of an atomizer. Some users prefer drippers because it is a more customizable experience. A user can modify the atomizer by changing the length or grade of the coil, the number of coils, and the power and resistance of the coil. Those factors affect vapor volume, temperature, and flavor, which make dripping a more customizable vaping experience. However, dripping requires the user to add more e-liquid after just two or three drags, and so is a less convenient method of vaping, especially for a user on the go.

There are several shortcomings of the prior art delivery methods that atomize those e-liquid solutions. For example, in prior art utilizing a tank for storage of the liquid for atomization, the tank volumes are often relatively large. Given that users often prefer to fill a tank with a large volume of liquid, it is inconvenient in the sense that a user must completely consume the liquid or must remove the liquid manually and replace it with a different liquid if they desire a different mixture. Furthermore, since it is difficult to completely remove a previous mixture from the internal components of a tank and atomizing apparatus, the mixtures can interact and create combinations that may be undesirable.

Drippers avoid the inconveniences of a storage tank, but they require more manual involvement by the user, who must remove the mouthpiece or other cap, add more e-juice from a dropper or other container, and reinstall the mouthpiece or cap between every two or three puffs.

for both tank and dripper devices, frequent users must keep e-liquid on hand in order to refill the device. E-liquids that contain liquid nicotine are poisonous if ingested. In addition to the inconvenience of storing and using multiple types of e-liquids, there are safety concerns as well. Some users have noted the danger of having colorful flavored liquids, which may be attractive to children, containing concentrated nicotine that is in fact harmful if ingested. The number of “dangerous exposures” to liquid nicotine more than doubled in the United States from 2013 to 2014, coinciding with a rise in popularity of vaping. See, for example, Moran, “Upstate New York boy, 1, dies after ingesting liquid nicotine,” New York Daily News, Dec. 15, 2014.

In addition to the convenience and safety concerns, prior art e-liquid delivery systems have other drawbacks as well. For example, aerosolizing e-liquid by heating may cause degradation and pyrolysis of some chemical species residing in the mixture. Flavors and active ingredients may be lost or chemically altered, yielding a suboptimal vaping experience. Controlling the temperature of a heating element provides some flexibility in tank chemical composition. However, some chemical components that would be desirable to inhale may be so heat-sensitive that they may degrade, conjugate, pyrolize, or otherwise become chemically altered even at the minimum temperatures required to vaporize them. Products of pyrolysis, for example, which may be undesirable to inhale, are carried by the vapor. Conversely, the greater the fraction of a desirable component that is chemically altered by the heat provided for its vaporization, the lesser the fraction, if any, of the native species that remains unaltered to be carried in the vapor stream. Examples of particularly heat sensitive species include thiosulfinates derived from plants of the Alliacea family.

### SUMMARY OF THE INVENTION

The present disclosure provides devices, systems, and methods that make it more convenient for a user to exchange the flavoring or physiological active species with ease and without mixing of previously loaded mixtures. It further provides devices, systems, and methods for introducing flavoring, nicotine, or other chemical species desired for consumption by the user into e-cigarette vapor without heating them, thus avoiding excessive pyrolysis or degradation to those ingredients. The disclosure provides flexibility to tailor a unique experience for each specific user. The disclosure further provides safer alternatives to current e-liquid containers and devices, making them less accessible to children, and making harmful exposure to liquid nicotine less likely in general.

The disclosure provides systems, devices, and methods wherein a solution of propylene glycol, vegetable glycerin, or both (pg/vg solution) is kept separate from the flavoring, nicotine, or other active ingredients. Thereby, the e-liquid that is heated and atomized by the hot coil consists solely of pg and vg, while the flavoring, nicotine, or other active ingredients are added to the vapor afterwards. The flavoring, for example, can be added to the vapor by means of the vapor passing through a cartridge containing the flavoring.

According to one aspect of the present invention, breathable fluid delivery device (or inhalation device) components are presented. In one aspect, the disclosure presents an auxiliary conduit attachment for coupling with electronic cigarettes whereby chemical species can be introduced into a fluid stream provided by a coupled parent electronic cigarette or apparatus capable of forming such a fluid stream as described herein.

According to another aspect of the disclosure, a tank is provided for use with a battery base of an electronic vaping device. The tank may include an atomizer. The tank may be factory-sealed, with a pre-loaded amount of e-liquid contained therein. The tank may be configured to prevent a user from opening the tank or otherwise extracting the liquid, except by atomizing it with a proper vaping device. The tank may be designed to prevent refilling with e-liquid, or in other words, to be disposable, such that when the e-liquid is expended, the tank is intended to be discarded.

According to another aspect of the present invention, a complete inhalation device, such as an electronic cigarette device, for delivery of atomized liquid and other functional chemical species is provided. The device is preferably a handheld assembly of components including a battery assembly, an electronic control circuit, at least one atomizing element, and a series of fluid conduits. The atomizing element may comprise any suitable mechanism for atomizing a liquid to be introduced into a breathable (generally interchangeable with “inhalable” for purposes of this description and claims) fluid stream.

The fluid conduits include an atomization liquid storage chamber, an atomization chamber, a suitable exchangeable fluid-permeable body, illustrated and described herein as a “cartridge” comprising a porous packing material for containing a functional liquid, and a mouthpiece. Cartridges generally referred to herein may include a casing for containing the porous packing material, which is desirable for retaining functional liquid within the cartridge and/or imparting a shape to certain packing materials. However, if the packing material is a solid, stiff, unitary mass, which may be termed a “pod” in the context of the invention, the casing is not essential to hold the shape of the pod, and may optionally be omitted despite the possible increased tendency of leaking or evaporation of functional liquid therefrom. In some embodiments, the device includes an atomizing element associated with the cartridge itself, to atomize and facilitate the introduction of the functional liquid containing in the cartridge into the breathable fluid stream, either in lieu of or in addition to an atomizer tank assembly connected in line with the cartridge.

The device further includes electronic components such as a battery, an electronic controller for directing a controlled voltage and current to other electronic components, a user display and/or input interface, a signal receiver component, electrically powered atomizing elements, a button/switch/other manual actuator, and conductive wires or other circuit elements. The electronic controller may include a processor, a memory, and/or one or more sensors, which enable the device to perform functions such as storing, recalling, and presenting to a user historical device state data or usage data, adjusting current or voltage in response to detected overheating of components or other predetermined device states, automatically actuating atomizing elements in response to sensing a user drawing breath from a mouthpiece, and actuating or “unlocking” atomizing elements in response to a passcode input.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

### BRIEF DESCRIPTION OF THE DRAWINGS

FIG. 1A is a cap device for use with a dripper.

FIG. 1B is a cross-section of a cap device for use with a dripper.

FIG. 2 is an exploded view of the cap device from FIG. 1A and FIG. 1B.

FIG. 3 is a transverse cross-sectional view of a fluid-permeable cartridge according to another aspect of the invention.

FIG. 4 is an end view of a fluid permeable end of the cartridge shown in FIG. 5.

FIG. 5 is an exploded cross-sectional transverse view of the cartridge shown in FIG. 5

FIG. 6A is a disposable atomizer tank.

FIG. 6B is a cross-section of a disposable atomizer tank.

FIG. 6C is an atomizer for use with the disclosed devices.

FIG. 6D is a base of an atomizer tank.

FIG. 6E is an exploded view of an atomizer tank.

FIG. 7 is an exploded perspective view of an exchangeable atomizer tank assembly according to another aspect of the invention.

FIG. 8 is an exploded perspective view of an alternative exchangeable atomizer tank assembly according to another aspect of the invention.

FIG. 9 is a transverse exploded plan view of a complete inhalation device incorporating a fluid permeable cartridge and variable valve system in accordance with an aspect of the invention.

FIG. 10 is an exploded perspective view of an exchangeable atomizer tank assembly according to another aspect of the invention.

FIG. 11 is a fragmentary transverse cross-sectional view of an inhalation device, illustrating an atomizer tank.

### DETAILED DESCRIPTION

Illustrative embodiments and exemplary applications will now be described with reference to the accompanying drawings to disclose the advantageous teachings of the present invention.

In this disclosure, relational terms such as first and second, top and bottom, proximal and distal, upper and lower, and the like may be used solely to distinguish one entity or action from another entity or action without necessarily requiring or implying an actual such relationship or order between such entities or actions. In this disclosure, the use of the term “proximal” with relation to the anatomy of the present invention may be used to distinguish the end of the disclosed assembly that is at the farthest end of the mouthpiece, while the “distal” end refers to the farthest end of the battery enclosure. In other words, the breathable fluid stream travels in a proximal-to-distal direction during normal use of the vaping devices disclosed herein. However, the relative positions and orientations of components or features of an inhalation device are described and depicted for illustrative purposes and are not required unless expressly stated.

In this disclosure, the terms “fluid,” “fluid stream,” and “fluid flow” may refer to any suitable fluid composition, including but not limited to pure air or air mixed with an atomized, volatilized, nebulized, discharged, or otherwise gaseous phase or colloidal aerosol form of a functional liquid or atomizing liquid described herein.

In this disclosure, the term “functional liquid” shall be understood to represent a chemical species, composition, or mixture thereof, which is intended to be volatilized, atomized, or otherwise introduced into a fluid stream that is in communication with said liquid. The functional liquid may be comprised of any single chemical species or combination of chemical species having desirable properties for enhancing an inhaled fluid stream and being suitable for adsorption upon or absorption into media suitable for use in the present invention. Furthermore, a functional substance in non-liquid form, which may for example be crystalline or otherwise solid, may be substituted for a functional liquid without departing from the scope of the invention.

In this disclosure, the term “atomizing liquid” shall be understood to represent a chemical species, or mixtures thereof, which is intended to be vaporized, nebulized, or otherwise introduced into and carried with a said fluid stream passing through the present invention. Furthermore, in extension of previous discussion, the atomizing liquid may emulate various physical characteristics of tobacco smoke upon atomization, such as a visible plume and/or the temperature, bulk, flavor, or other organoleptic qualities of the inhaled stream. Furthermore the atomizing liquid may also act to enhance the solvation characteristics of the fluid stream or act to deliver heat energy to another material or substance, such as through condensation. According to the present invention, the atomizing liquid may include propylene glycol or vegetable glycerin, for example. Transition of a chemical species from liquid form to a gaseous phase or nebulized phase is commonly facilitated by rapid heating on a resistive coil, or nebulization via a vibrating film, plate, or reed. It is to be appreciated that the present invention is not limited to utilization of the stated methods and/or chemicals, but may employ any suitable mechanism capable of transitioning a liquid into a gaseous or nebulized phase to be carried in a fluid stream providing the desired functions as stated or implied.

The present invention relates to breathable fluid delivery apparatuses, such as electronic cigarettes and other inhalation devices, including an exchangeable fluid permeable cartridge containing a liquid mixture of chemical species to be introduced into a breathable fluid stream passing through the cartridge, which may comprise air in combination with atomized liquid, vapor, or both. With respect to the phrase “introduced into a breathable fluid stream,” one skilled in the art will understand the term “breathable” in a broad sense of being comfortable for a human user to inhale intermittently, as in the ordinary use of electronic cigarettes, vaping pens, and similar devices. In the context of the invention, the term “breathable fluid stream” includes but is by no means limited to pure air and substances similarly suitable for breathing continuously for extended time periods. The exchangeable fluid permeable cartridge may be disposed in an auxiliary conduit attachment substituting for the mouthpiece of an atomized liquid delivery system (such as an existing electronic cigarette).

The present invention also discloses disposable and exchangeable tanks for e-liquids. In some embodiments, a tank comes factory-sealed with e-liquid already inside. In other embodiments, a user can inject or otherwise insert an e-liquid of choice into the tank before use. In some embodiments, the e-liquid comprises propylene glycol (pg), vegetable glycerin (vg), or both. In some embodiments the e-liquid further comprises natural or artificial flavorings, nicotine, or other functional substances. In some embodiments the pg/vg liquid is contained in the tank, while the flavorings or other functional substances are contained in a cartridge downstream of tank, whereby heating and atomization occur only on the pg/vg, and the flavorings or other functional substances are mixed with the aerosolized pg/vg later.

In a preferred embodiment, a tank of the present invention comprises a substantially cylindrical reservoir containing e-liquid. The reservoir has an outer wall which may be translucent to allow visualization of the quantity of e-liquid remaining in the tank. The tank also has an inner wall separating the reservoir from a central bore that is configured to receive an atomizer there through. The inner and outer walls are joined together at a distal end of the tank, closing off the tank and forming a trough bound by the inner and outer walls. On the proximal end, a metal cap seals the trough, making it fluid-tight. The base is connected to the atomizer and has a connector, which is compatible with battery units, such as via a 510 connector.

As is known in the art, the atomizer comprises a metal coil and a wicking material extending into the tank, capable of drawing e-liquid into the coil. When in use the coil is electrically connected to the positive and negative terminals of the battery unit. The coil is capable of receiving power from the battery and heating the e-liquid soaked up by the wick, thereby atomizing the liquid. The atomized liquid can be drawn through the breathable fluid stream and out the distal end of the central bore, which can be connected to a separate mouthpiece. Optionally the tank can have a mouthpiece permanently affixed.

The tank may be constructed from resilient materials such as plastics and metals, that are capable of withstanding the heat and pressures that would commonly be present in a vaping apparatus. Nevertheless, the tank may be a single-use device, such that when the e-liquid is used up, the empty tank is disposable and/or recyclable. In some embodiments the tank is constructed to prevent it from being refilled by usual means of refilling a tank, as known in the art. For example, many prior art tanks can easily be manually pulled apart and more e-juice inserted via a dropper or injector. In certain embodiments, the tanks of the present disclosure can be refilled by the manufacturer inserting a needle through a fill port in the base.

In accordance with an aspect of the present invention, an apparatus for delivering a functional liquid in a breathable fluid stream for inhalation is provided. The apparatus may be an attachment for a breathable fluid delivery device, comprising a mouthpiece, which may be removable, including a distal opening in fluid communication with a proximal opening; and a cartridge comprising a fluid-permeable packing material, configured for drawing a breathable fluid through the packing material by inhaling through the mouthpiece. The phrase “in fluid communication” will be understood to refer broadly to components or features of the device disposed in the path of a common fluid channel or conduit, without limitation to components that are necessarily directly adjacent to one another.

The packing material may contain a functional liquid disposed to be introduced into the fluid stream as the fluid stream passes through the packing material and to be carried in the fluid stream in a breathable form when the fluid stream passes out of the mouthpiece through the distal opening. The functional fluid may provide a flavor, a recreational and/or medicinal drug effect, or other desired effect when the breathable fluid stream is inhaled.

A cartridge suitable for use with embodiments of the invention may consist solely of a fluid permeable packing, or may further comprise additional elements. For example, the cartridge may include a casing covering at least a portion of the packing. Where present, the casing may include open ends or fluid permeable ends that nonetheless provide support to prevent the packing from falling out of or being removed from the casing. Whether the casing includes open ends or fluid-permeable ends that support/retain the packing, the ends of the casing may be sealable for storage or shipping by a removably adhered film. The packing itself may also take different forms, including a single solid porous body or pod, or a mass of fibers, particles, or grains having spaces there between, functioning to permit fluid passage, similarly to the pores of a single porous body.

The cartridge may be retained in a housing. In some embodiments the housing and cartridge are manufactured and made available to the consumer as a single unit, which is not intended to be opened, and the liquid not intended to be removed except via atomization and regular use with a vaporization device. In such an embodiment the cartridge and housing together are a single exchangeable and disposable unit. The cartridge housing includes a proximal opening in fluid communication with the breathable base fluid (such as atomized or aerosolized pg/vg) and a distal opening in fluid communication with the proximal mouthpiece opening, and the housing may include a structure to support the cartridge in a position in which a portion, such as a proximal end, of the cartridge is in fluid communication with the proximal housing opening and a portion, such as a distal end, of the cartridge is in fluid communication with the distal housing opening. The housing may be removably connected to a fluid delivery device, for example via a coupling adaptor, the fluid delivery device being configured to deliver a stream of the breathable base fluid into the proximal housing opening. Coupling adaptors according to the invention may or may not be interchangeable with existing mouthpieces of existing breathable fluid devices. Such coupling adaptors are well known in the art, such as the 510/eGo threading adaptor, as used on the eGo Twist available from Joyetech USA, Inc. (Irvine, Calif.).

Preferably, the apparatus or attachment includes a flow channel in which the packing is disposed, the flow channel including a wider portion adjacent at least one of the distal and the proximal end of the cartridge, and a narrower portion adjacent the wider portion and extending therefore in a direction away from the cartridge. Advantageously, to maximize the surface area available for inflow of breathable fluid through the cartridge, a spacer feature abuts the cartridge to maintain an axial distance between the cartridge and the narrower channel portion. The spacer feature may comprise a separate annular member, or it may be integrated into, for example, a cartridge casing or housing. The spacer may have a uniform longitudinal dimension, or it may have a tapered dimension, so that no part of the proximal/upstream/inlet area of the cartridge is covered so as to block inflow of breathable fluid.

Some embodiments of the present disclosure can be used in conjunction with exchangeable cartridges, cartridge housings, and similar units as disclosed in related U.S. patent application Ser. Nos. 14/203,560 and 14/333,920, both of which are incorporated by reference herein in their entireties.

In some embodiments the cartridge described herein comprises a fluid-permeable packing material containing an absorbed or adsorbed quantity of a functional liquid configured to be introduced into a breathable fluid stream flowing through the cartridge. In other embodiments the cartridge contains a functional liquid not absorbed or adsorbed into a packing material. The cartridge may further include a fluid-impermeable covering disposed over at least a portion of the outer surface area of the packing material. Such a covering may comprise a wall structure generally surrounding the packing material, with or without one or two fluid-permeable ends joined to the wall structure to fully retain/cover/encase the packing. To facilitate shipping and/or extended storage, the covering may include a fluid-impermeable film material removably fixed over the ends of the covering to seal the packing material and functional liquid within the covering for storage and/or transport prior to use of the cartridge. Alternatively, the covering may also accept sealing caps that may be compressed onto the ends, such as plastic caps, which could act to seal the cartridge when it is not in use or in storage.

Suitable film materials may include, without limitation, metal foil, BoPET (Biaxially-oriented polyethylene terephthalate), and plastics, and the film may be affixed by adhesive and/or heat-fused wax. Packing materials may be selected from among cotton, foam, fibrous media, stacked thread, stone, synthetic porous media, and any other materials having the desired adsorption/absorption and fluid permeability properties.

In accordance with yet another aspect of the invention, a conduit assembly for a breathable fluid delivery device is provided. In particular, a fluid-permeable cartridge is disposed in a main fluid channel for inhalation delivery of a breathable fluid stream, the cartridge containing a functional liquid disposed to be introduced in a breathable form into a breathable base fluid flowing in the main fluid channel through the cartridge. A source of the breathable base fluid is connected in fluid communication with an upstream end of the cartridge—this may be any reservoir of or component or system for delivering a breathable base fluid, including but not limited to a fluid tank and a heating coil or vibration element, for example, where such a tank and atomizing element are sometimes collectively termed a “cartomizer” or a “clearomizer,” as are well known in the art. A mouthpiece is connected in fluid communication with a downstream end of the cartridge, and an adjustable valve system may be incorporated in the assembly for adjusting the flow impedance of a bypass channel passing around the cartridge relative to the flow impedance of a portion of the main channel extending through the cartridge.

In accordance with aspects of the present disclosure, breathable fluid delivery device components are presented. In one aspect, the disclosure presents an auxiliary conduit attachment for coupling with electronic cigarettes whereby chemical species can be introduced into a fluid stream provided by a coupled parent electronic cigarette or apparatus capable of forming such a fluid stream as described herein.

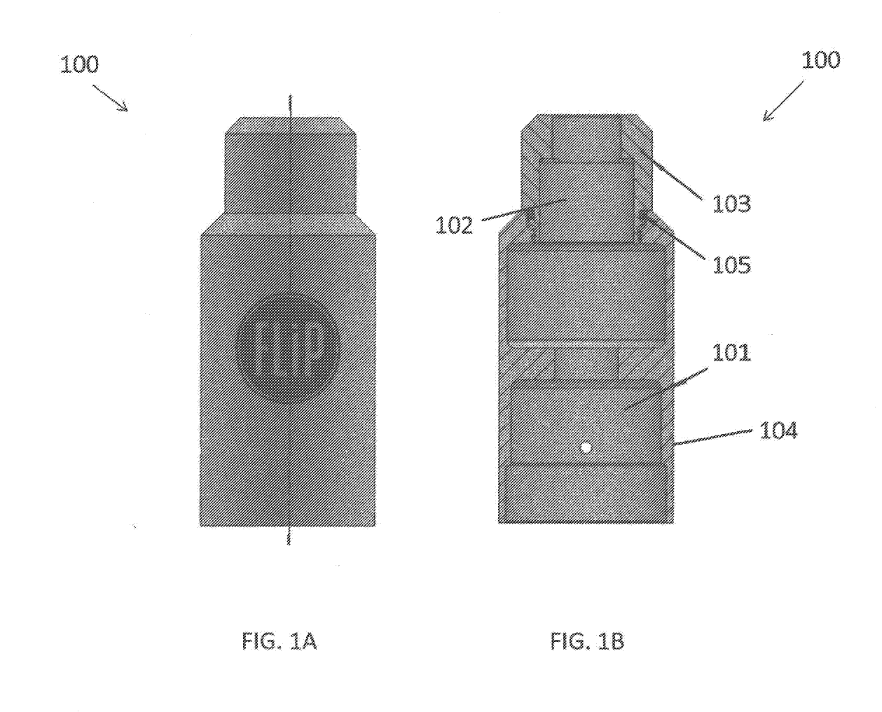

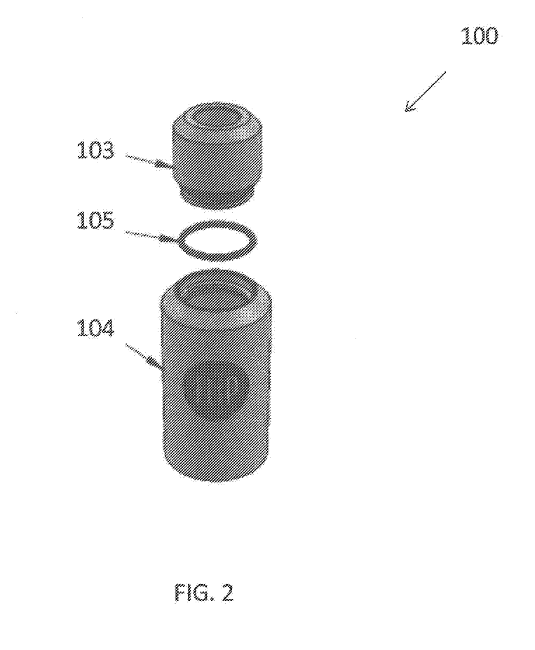

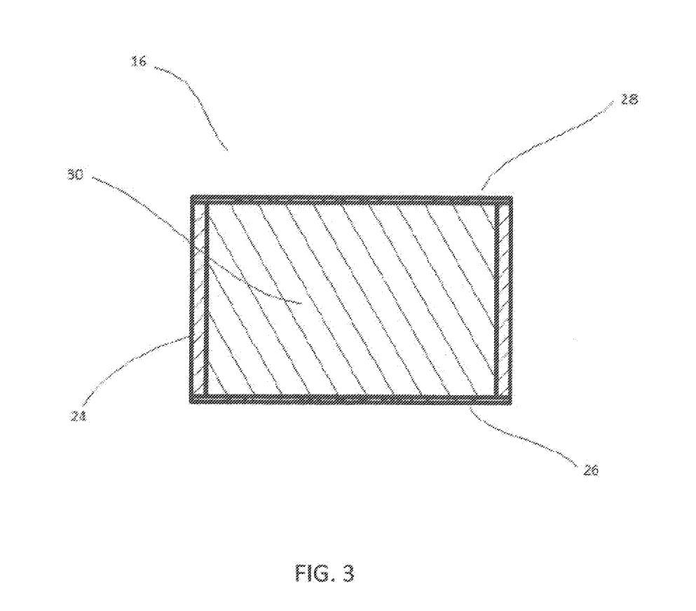

In accordance with a first aspect of the present disclosure, with reference to an embodiment thereof illustrated in FIG. 1, an auxiliary conduit attachment 100 for coupling with breathable fluid delivery devices such as electronic cigarettes will now be described. The attachment or cap 100 is designed for use with a dripper base, such as the Patriot RDA, available from Vapor Labs, Inc. (Garden Grove, Calif.). The cap 100 is attachable at its proximal end to a dripper base and fits snugly thereon. The atomizer of a dripper base unit (not shown) fits in the cavity 110 formed at the proximal end of the cap 100. Preferably e-liquid containing only pg and/or vg is dripped onto the coils, and the atomized vapor then travels distally through the lumen of the cap towards the chamber 120 containing a cartridge (shown in FIG. 3 and described in detail below). A cartridge or “pod” may contain a flavor and/or other functional substance. The distal portion 130 of the cap 100 has a threaded region that attaches to a threaded region of the proximal portion 140. A user can inhale through the distal portion, or can attach a mouthpiece. In other embodiments not shown, the cap 100 can attach in line with additional housings with cartridges. The FIG. 2 shows an exploded view of the cap device of FIG. 1. An o-ring fits between the proximal portion 140 and the distal portions 130, creating a seal.

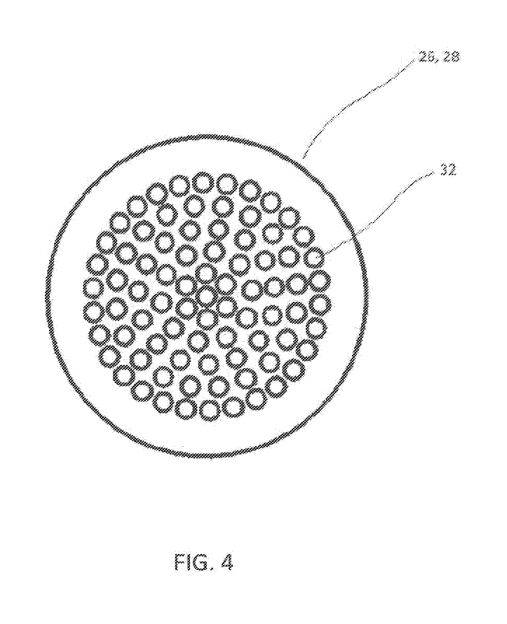

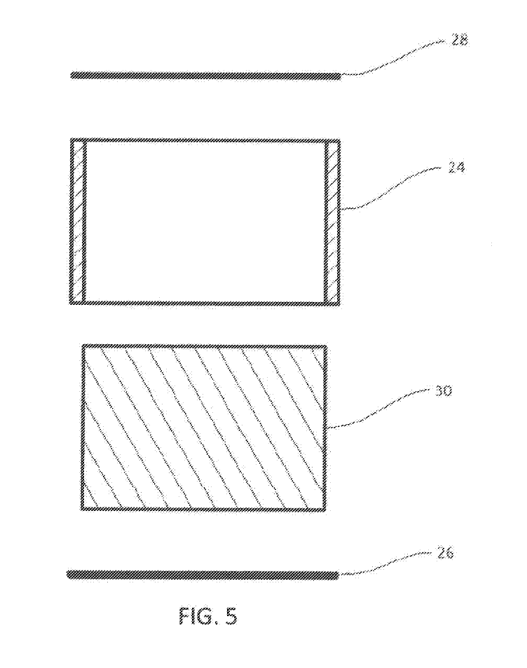

As shown in FIG. 3, the cartridge 16 may comprise a casing 24, fluid permeable ends 26 and 28, and a fluid permeable packing 30, which may for example be a single porous body or a mass of fibers, coarse grains, or particles of material including spaces there between, even if the individual fibers, grains, or particles are themselves non-porous. Casing 24 is preferably a tube-like structure having a hollow channel spanning its length and open ends. At the ends of casing 24, fluid permeable ends 26 and 28 are connected or attached to allow the passage of a fluid through the casing channel and packing 30, while providing containment of packing 30 inside casing 24. Fluid permeable ends 26 and 28 may be, but are not limited to, mesh screens or perforated sheets having arrayed apertures 32, as illustrated in FIG. 4. As an additional aspect, one or both of ends 26 and 28 may be constructed from the same piece of material as the casing 24, but with fluid permeable properties. Ends 26 and 28 may be constructed to permit sealing with a thin film or the like (not shown) for storage and packaging of cartridge 16. This film may for example be metal foil, plastic, or other comparable materials that could be fixed to ends 26 and 28 via an adhesive, heat fused wax, or other comparable method. FIG. 5 shows an exploded view of the cartridge 16 of FIG. 3.

Packing 30 may be composed of any suitable material that is fluid permeable and does not pose an inhalation health risk. Suitable materials for packing 30 include cotton, foam, stacked thread, porous stone, synthetic porous media, or any other material which is capable of adsorbing or absorbing the desired chemical species in liquid phase. Packing 30 is configured to accept a functional liquid, while still maintaining fluid permeable properties. In particular, packing 30 should be capable of holding a reasonable quantity of the functional liquid before becoming saturated, to avoid the need for frequent replacement of cartridge 16.

In certain preferred pairings of a packing material of packing 30 with a functional liquid, the surface chemistry of the packing material favors the adsorption of the functional liquid in order to improve saturation and functional liquid load. For example, a porous media having a surface chemistry that is hydrophilic may better saturate with hydrophilic functional liquids. A sintered porous plastic has proven to be a particularly effective porous medium, due to its tendency to force an air flow to spread generally evenly across its entire cross-sectional area, thereby exposing a greater volume of air to the functional liquid. This widening/dispersion of the air stream is believed to be largely due to a significant flow resistance produced by the material, as evidenced by a noticeable pressure drop across the length of cartridge 16 when packed and saturated. Other media may exhibit similar flow properties with similar effects.

Functional liquids that may be advantageously contained in cartridges according to the invention include, without limitation, esters, acetate esters, alcohols, acids, lactones, carbonyls, terpenes, thiols, saturated and unsaturated thiosulfinates, hemiterpenes, monoterpenes, sesquiterpenes, diterpenes, sesterterpenes, triterpenes, sesquiterpenes, tetraterpenes, polyterpenes, norisoprenoids, and derivatives thereof, such as terpin hydrate, a derivative of turpentine; natural flavor compounds such as those often found in fruits, including but not limited to: Gamma Decalactone, Gamma Octalactone, Butyric Acid, 2-Methyl Butyric Acid, Proprionic Acid, Isovaleric Acid, Isobutyric Acid, Cinnamic Acid, Phenethyl Alcohol, Ethyl Butyrate, Ethyl Isobutyrate, Ethyl-2-Methyl Butyrate, Ethyl Isovalerate, Methyl Cinnamate, Ethyl Proprionate, Ethyl Hexanoate, Isoamyl Isovalerate, Phenethyl Acetate, (Z)-3-hexenal, beta-ionone, hexanal, beta-damascenone, 1-penten-3-one, 3-methylbutanal, (E)-2-hexanal, 2-isobutylthiazole, 1-nitrophenylethane, (E)-2-heptenal, furanones, 2,5-dimethyl-4-hydroxy-3(2H)-furanone, methyl 2-methylbutanoate, ethyl 2-methylpropanoate, methyl hexanoate, methyl butanoate, trans-2-hexenal, ethyle-2-methylbutanoate, ethyl butanoate, trans-2-hexenol, hexyl acetate, hexyl butanoate, 1-butanol, 1-hexanol, cis-3-hexenal, cis-3-hexeol, cis-3-hexenyl acetate, ethyl hexanoate, propyl 2-methylbutanoate, 2-methyl-1-butanol, benzyl alcohol, 1-octanol, 2-phenylethanol, 1,3-oct-5(Z)-enediol, 1,3-octanediol, 4-vinylguaiacol, eugenol, 2-methylbutanoic acid, 4-hydroxyphenylacetic acid, 3-hydroxy-beta-damascone, 4 hydroxy-3-methoxyphenylacetic acid, 3-oxo-alpha-ionol, vomifoliol, 3-Oxo-ü-ionol, dehydrovomifoliol, roseoside; and/or natural flavor compounds such as those found in vegetables, including but not limited to: dimethyl sulfide, thiosulfinates, disulfides, poly-sulfides, 2-propene-1-sulfinothioic acid S-2-propenyl ester (allicin), methanesulfinothioic acid S-2-propenyl ester, 2-propene-1-sulfinothioic acid S-(E,Z)-1-propenyl ester, 2-propene-1-sulfinothioic acid S-methyl ester, Linoleic acid, (E)-2-nonenol, (E)-2-nonenal, (Z)-3-nonenol, (Z)-3-nonenal, C9 Carbonyls, (Z,Z)-3,6-nonadienal, (E,Z)-2,6-nonadienal, 3-methylbutanoates, 2-phenethyl esters, 2-phenethyl 3-methylbutanoate, (E)-2-hexenyl 3-methylbutanoate, benzyle 3-methylbutanoate, (E)-2-hexenyl 3-methylbutanoate, benzyl 3-methylbutanoate, methyl 3-methylbutanoate, butyl 3-methylbutanoate, 3-methylbutanoate, butyl 3-methylbutanoate, 3-methylbutyl 3 methylbutanoate, (E)-2-pentenyl 3-methylbutanoate, 2-phenethyl hexanoate, sesqunterpene alcohol, cubenol, phthalides, 3-butylphthalides, 3-butyl-4,5-dihydrophalide, cis and trans forms of 3-butyl-3a,4,5,6-tetrahydrophthalide, (Z)-ligustilide, 1-(E,Z)-3,5,-undecatriene, sesquinterpene hydrocarbons, alpha-copane, alpha-muurolene, alpha-calacorene, cadinenes, 2-acetyl-1-pyrroline, 2-ethyl-3,6-dimethylpyrazine, acetaldehyde, 3-methylbutanal, 4-vinylguaiacol, 2-acetylthiazole, 2-acetyl-2-thiazoline, 2-(1-hydroxyethyl)-4,5-dihydrothiazole, 2,5-Dimethyl-4-hydroxy-3(2H)-furanone, hydrogen sulfide, methanethiol, ethanethiol, octa-1,5-dien-3-one, linolool, (E,E)-deca-2,4-dienal, p-mentha-1,3,4-triene, myrcene, 2-sec-butyl-3-methoxypyrazine, myristicin, (E,E)-deca-2,4-dienal, (Z)-dec-6-enal, Beta-phellandrene, (Z)-hex-3-enal, (Z)-hex-3-enol, (Z)-hex-3-enyl acetate, vanillin, menthol, methyl salicylate, 3,7-guaiadiene, delta-cadinene, cannabinoids, nicotine, caffeine, citicolene, and taurine. The current invention may also employ the vast array of melanoidins, a class of chemicals produced by Maillard reactions, wherein amino acids and reducing sugars are heated together to produce complex compositions of chemicals derived therefrom. In addition, extracts from plants and other biological materials may be utilized. Pharmaceutical inhalation delivery drugs may also be utilized, such as Ciclesonide, Cromolyn Sodium, Ipratropium Bromide, Nedocromil Inhalation, Albuterol Sulfate, Triamcinolone Acetonide, Albuterol Sulfate, Levalbuterol Tartrate, Flunisolide Hemihydrate, Fluticasone Propionate, Salmeterol, Fluticasone Propionate, Paclitaxel, Salmeterol Xinafoate, Metaproterenol Sulfate, Beclomethasone Dipropionate HFA, Beclomethasone Dipropionate Monohydrate, Ribavirin, N-acetyl-L-cysteine, Loxapine, Insulin, Pirbuterol, Budesonide, Formoterol Fumarate Dihydrate, Methacholine Chloride, Mometasone Furoate, Pentamidine Isethionate, Dornase alfa, Iloprost, Tobramycin, Fluticaone Propionate, Arformoterol Tartrate, Idarubicin, Levalbuterol.

As an alternative to cartridge 16 as illustrated, a cartridge within the scope of the invention may consist of only an open ended casing and a fluid permeable packing material residing inside the casing, such as a porous body, a quantity of particulate material, or a mass of fibers. The embodiment may contain the packing partially or completely saturated with a said functional fluid and the cartridge as a whole may be exchangeable in the same fashion as that of the disclosed embodiment consisting of casing 24, packing 30, and fluid permeable ends 26, 28.

In another embodiment, the cartridge may consist solely of a packing material, which may not necessarily have an outer casing or fluid permeable ends. The porous material may be partially or completely saturated with a functional fluid and may be exchangeable in the same fashion as that of the disclosed embodiment having casing 24 and fluid permeable ends 26, 28.

In certain embodiments the cartridge is pre-filled with flavoring or a functional substance during manufacturing. In other embodiments, a user can insert a substance into the pod. The pod may be single-use or reusable. In some embodiments multiple pods can be used together or “stacked” on top of each other to allow customizability for the user. Other embodiments of the device may include powdered nicotine or other functional substance in the pod or elsewhere in the breathable fluid conduit, such that those additional substances can be interchangeably added into the fluid stream.

Individual parts of attachments or fluid delivery devices according to the invention may be constructed out of any suitable material that permits ease of use thereof, durability, safety, and ease of manufacturing. In preferred embodiments, components are generally composed of a relatively hard, durable, and non-corrosive material, such as stainless steel, aluminum, brass, graphite, ceramics, silicon carbide, certain plastics or other suitable materials. Plastics used for components of an inhalation device according to the invention should generally be highly chemically resistant, as some functional fluids, such as certain alcohols, have been shown to cause degradation of certain existing plastic mouthpieces and polycarbonate parts. Suitable plastics may include silicones, thermoplastic elastomers/TPEs, Santoprene®, polytetrafluoroethylene (PTFE), polyaryletherketone family plastics, such as PEEK (polyether ether ketone), PVDF (polyvinylidene difluoride), PVC (polyvinyl chloride), CPVC (chlorinated polyvinyl chloride), Nylon®, Teflon®, HDPE (high density polyethylene), LDPE (low density polyethylene), Acetal, ABS (Acrylonitrile butadiene styrene), Halar®, Fluorosint®, Polypropylene, Polysulfone, PPS (polyphenylenesulfide), Torlon®, UHMW (ultra-high-molecular-weight polyethylene), CAB (cellulose acetate butyrate), Ertalyte®, Nylatron®, Acetron®, TIVAR®, Proteus®, and Sanalite®.

In preferred embodiments, materials used do not pose a significant health risk to users under normal use conditions, and should be selected to be compatible with the functional fluids used. For example, some functional fluids suitable for use according to the invention, including alcohols and terpenes, are corrosive to certain materials that are otherwise desirable for use in the devices of the invention. Polycarbonate is an example of a clear, hard plastic that may be advantageously used for device components, so long as it is not exposed to certain functional fluids that may damage it.

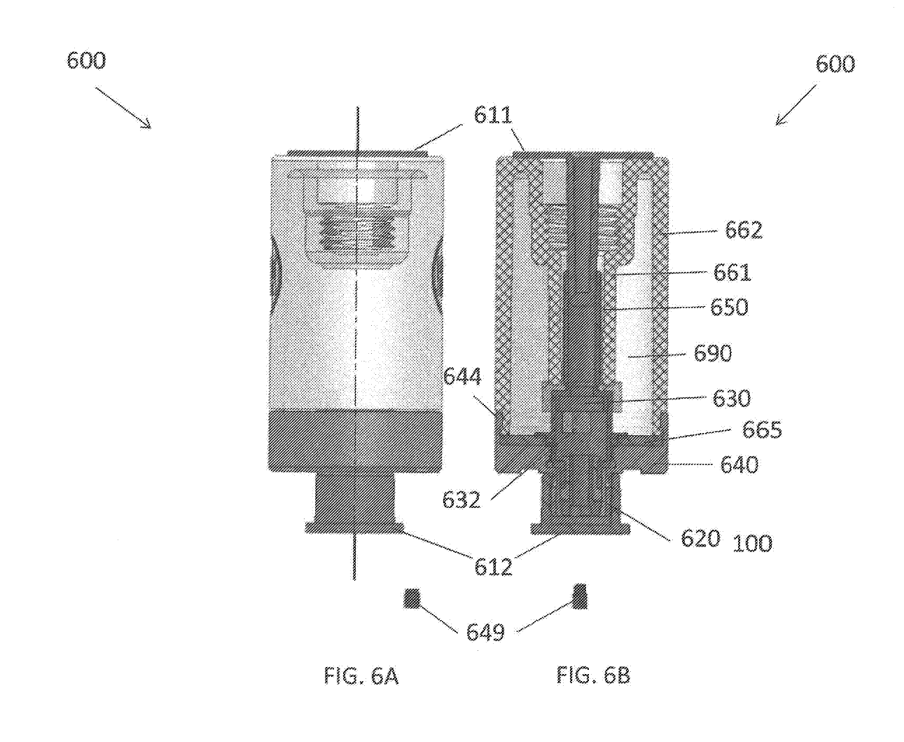

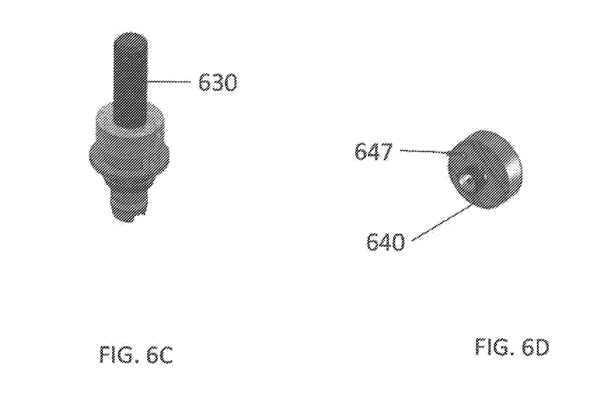

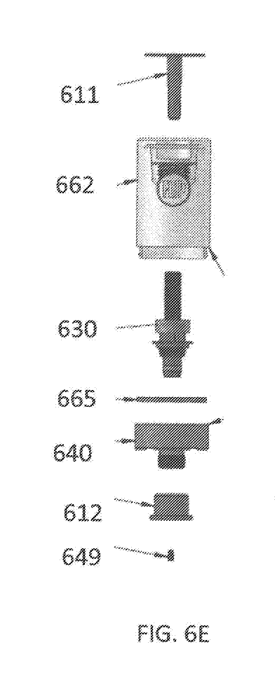

FIGS. 6a and 6b shows an atomizer tank 600 for holding e-liquid. FIG. 6a shows the tank 600 as assembled for shipment and delivery. The tank 600 has a disposable top seal 611 and a disposable bottom seal 612 for protecting the internal parts of the tank prior to delivery to a user. FIG. 6b is a cross-sectional view showing the internal parts of the tank. At the proximal end, the disposable bottom seal covers the connector 620 portion of the base 640. In this case the connector is a “510” connector, but in other embodiments a different type of connector can be used. The connector is capable of connecting with a battery unit (not shown), which powers the atomizer 630. The atomizer shown is a Kanger Coil Unit, available at www.szkanger.com, as shown in another view in FIG. 6C. The atomizer screws into the internal threads of the base 640. The atomizer 630 sits within the central bore 650 defined by the internal wall 661 of the tank, and the disposable top seal 611 fits into the distal portion of the atomizer coil unit 630. The outer wall 662 of the tank is in contact with a gasket 665, which forms a seal between the base and the outer wall 662, creating a fluid-tight trough 690 inside the tank 600. The wick 632 of the atomizer extends into the trough area 690 of the tank, so that it can be in contact with the e-liquid contained therein. The individual components of the tank are shown in an exploded view of the tank in FIG. 6E.

To assemble the tank 600, the manufacture inserts the disposable gasket 665 into the disposable base. Then the atomizer 630 is screwed into the base 640 until the threads bottom out. The tank is pressed onto the base 640 until the snap fit 644 is engaged. The top seal 611 is inserted into the distal portion of the tank, and the bottom seal 612 is inserted over the threads of the connector 620 portion of the base.

FIG. 6d shows a view of the base unit 640. The base includes a fill port 647. After assembly the tank is filled with pg/vg by inserting a needle through the fill port 647 in the base. The needle punctures the self-healing gasket 665. A plug 649 is inserted into the fill port.

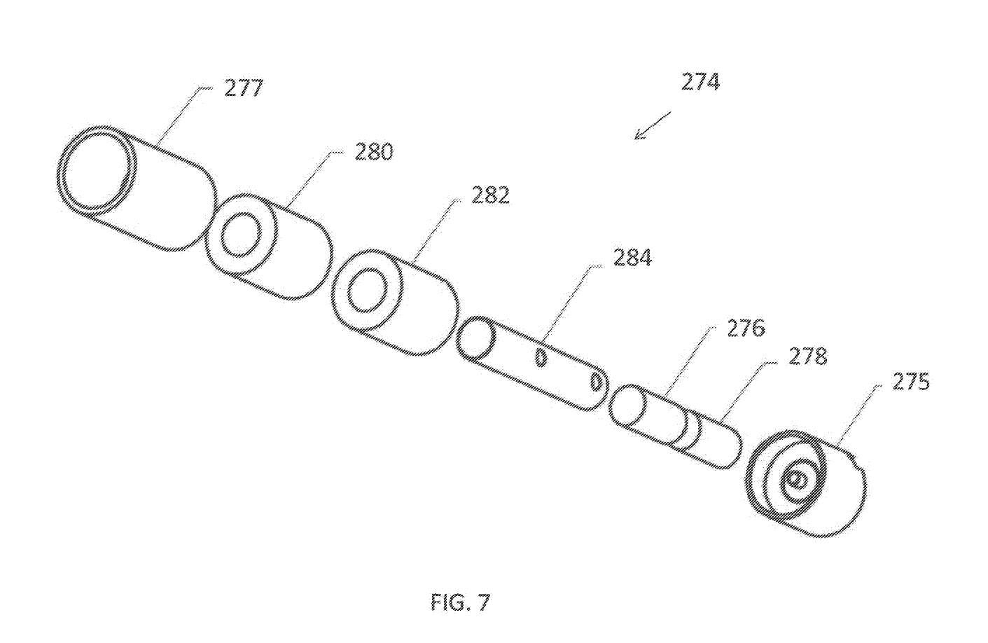

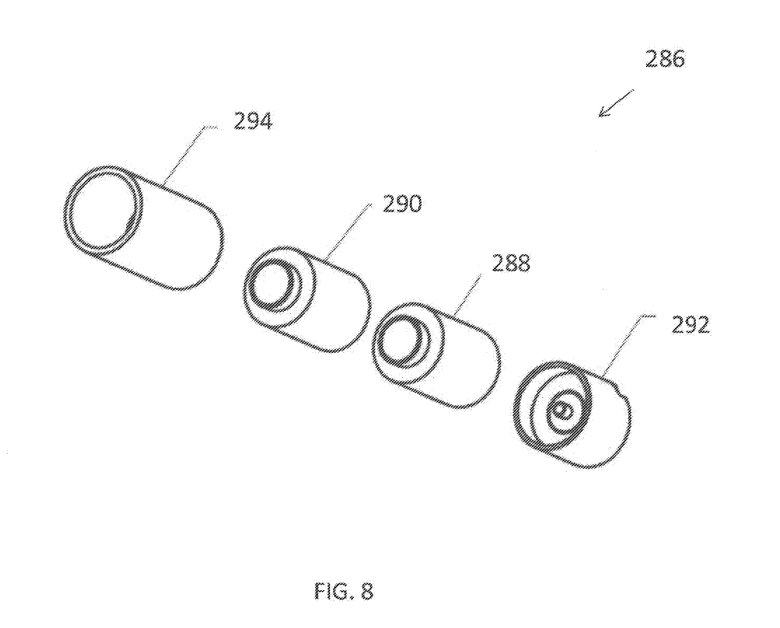

FIGS. 7 and 8 depict alternative embodiments of exchangeable atomizer tanks, with separable and built-in atomizing elements, respectively. Advantageously, exchangeable atomizer tanks facilitate alternation between atomizing liquids (such as liquids having different flavors, for example) by switching out one partially used tank for another, as well as permitting disposal and replacement of relatively cheap to manufacture tank components, typically made of plastic, of an atomization system with new, optionally prefilled components.

Referring to FIG. 7, an exchangeable atomizer tank assembly 274 is depicted, arranged between a battery/controller connector 275 and another assembly component 277 (which may for example be an exchangeable fluid-permeable cartridge housing), in which two distinct atomizing elements 276 and 278 are placed in line and in electrical communication with each other. Exchangeable atomization reservoirs 280 and 282 can slidingly fit over atomization chamber 284 and provide atomization liquid to the individual atomization elements. Flow of the liquid to the atomization elements may be initiated by piercing a liquid tight septum (not shown) in atomization reservoirs 280, 282, for which a suitable piercing tool (not shown) may be movably incorporated into a component of or otherwise provided with assembly 274. The septum may for example be any suitable film or membrane composed of a suitable material such as silicone, rubber, plastic, or metal foil, covering a flow channel inside the reservoir. Reservoirs 280, 282 may be exchangeable and disposable to allow a user to exchange atomization liquids without the need to clean or remove residues from previous liquids used in reservoirs 280, 282. Additionally, reservoirs 280, 282 preferably employ a mechanism for liquid delivery to the atomization elements that permits a user to remove an atomization reservoir without liquid leaking at any reservoir liquid level or state, such as by a spring-loaded valve mechanism (not shown) that is biased to a closed state when 280, 282 are removed from the assembly.

Turning to FIG. 8 an alternative exchangeable atomizer tank assembly 286, including distinct, preferably exchangeable and disposable atomization reservoirs 288 and 290 having associated within them distinct atomization chambers and atomization elements (not shown), arranged between a battery/controller connector 292 and another assembly component 294. In such an arrangement, atomization reservoirs 288 and 290 may be assembled adjacent each other in series as depicted, or alternatively, having one or more other components disposed between them (not shown), such as an exchangeable fluid permeable cartridge according to the invention, with or without an associated atomizing element. Reservoirs 288 and 290 are electrically connected in series or in parallel as desired. Electrically resistive elements associated with reservoirs 288 and 290, or interposed between them, may be provided to permit a user to selectively choose the amount of electrical current permitted through each atomization element in each reservoir. Such resistive elements (not depicted) could be separate and exchangeable elements that are placed in between each reservoir in order to permit a user to selectively control the atomization quantities provided from each atomization element. Reservoirs 288 and 290 are preferably in fluid communication with each other, either in series or parallel, to deliver atomizing liquid from each reservoir into a breathable fluid stream passing through an inhalation device according to the invention.

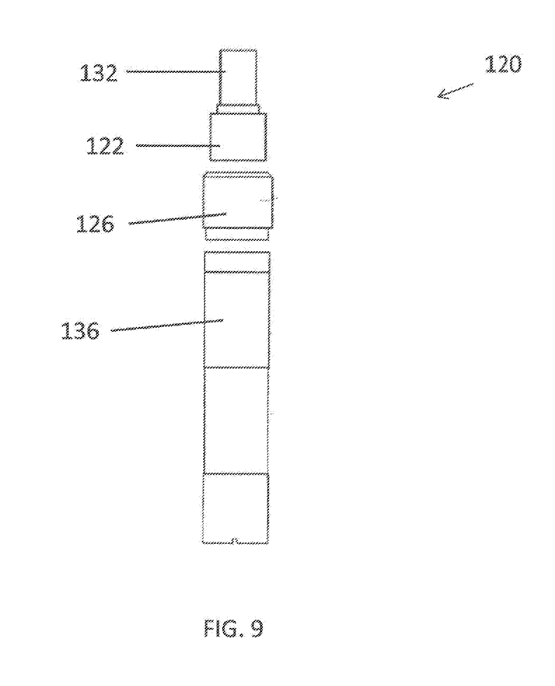

According to one example, FIG. 9 shows a schematic illustration of a complete inhalation device 120 incorporating the exchangeable porous cartridge and disposable tanks contemplated by the present disclosure. Inhalation device 120 includes a fluid-permeable cartridge component 122, and a breathable fluid delivering disposable tank 126. The tank 126 contains e-liquid such as a mixture of propylene glycol and vegetable glycerin and an atomizing element such as a heating coil, vibrating element, or the like (not shown). The tank 126 connects to a battery unit 136 to power the atomizer. When the device is in use, the atomized e-liquid, which in this case contains no flavoring or nicotine, travels through the cartridge and out the mouthpiece 132. In such an embodiment, the flavoring and active ingredients are not exposed directly to the atomizer. In other embodiments, a tank containing e-liquid with flavoring and/or active ingredients can be used with or without the additional cartridge. In such an embodiment, active ingredients such as liquid nicotine are safely confined in the sealed tank.

In accordance with other aspects of the invention, various embodiments of a complete electronic cigarette assembly, advantageously incorporating exchangeable porous cartridges according to the invention and/or other aspects of the present invention, will now be described in greater detail. The disclosure presents an electronic cigarette assembly designed to emulate the sensory experience of smoking a tobacco cigarette, while also being equipped with a system for introducing a functional liquid into the fluid stream passing through the assembly.

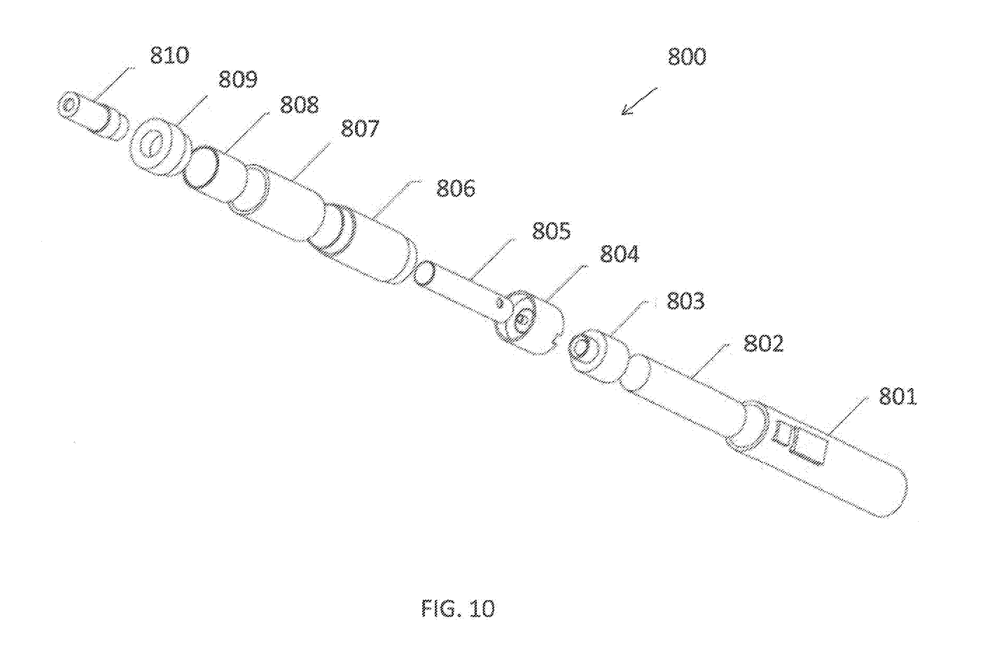

FIG. 10 shows an exploded view of an inhalation system 800 utilizing devices of the present disclosure. The system 800 is a nonlimiting example of how devices of the present invention can interact. Other configurations would be apparent to those skilled in the art. At the proximal end of the system is a battery pack including a battery pack casing 801, a battery 802, and a connection unit 803 for connecting with an atomizer. The tank portion comprises a connective base 804 and an atomizer 805 containing a coil (not shown) and a wicking material (not shown). The shell 806 of the tank forms a fluid-tight seal with the base 804, or optionally with a gasket (not shown) between it and the base. Distal to the tank 806 is an embodiment of a two-part cartridge housing 807/809 and a cartridge 808 according to the present disclosure. At the distal end is a mouthpiece 810.

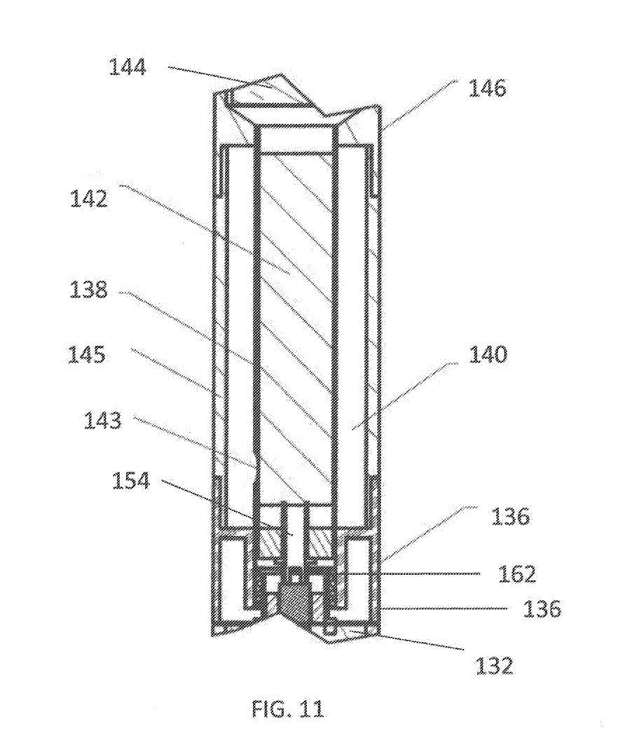

FIG. 11 shows an atomizer housing 138 connected between a battery/controller housing 136 and cartridge housing cover 146 and contains a single atomizing element 154. However, multiple atomizing elements may optionally be housed within a single atomizer housing. Other alternative embodiments of an atomizer housing may accept (or consist of) single or multiple exchangeable fluid permeable cartridges. Alternatively, an inhalation device may employ multiple atomization chambers and mechanisms, each adapted for atomization either of an atomizing liquid by itself or of an atomizing liquid and a functional liquid exclusively or simultaneously. Furthermore, the atomization of one or both of these liquid types may occur in distinct and separate atomization chambers, or in a combined atomization chamber. In other possible variations, an atomizing liquid and a functional liquid may undergo atomization in one chamber, a plurality of distinct chambers, in separate and distinct chambers simultaneously, in separate and distinct chambers at different times, both together in distinct chambers in addition to a separate chamber having one or both liquids, or both together combined and simultaneously atomized in separate and distinct chambers.

Atomizing element 154 is electrically connected to battery 132 by a contact pin 162 associated with atomizer housing 138 and atomizing element 154 contacting controller 134, thus providing a controlled amount of current and voltage to atomizing element 154. In turn, current flows from atomizing element 154 through conductive portions of atomizer housing 138 and of battery/controller housing 136 to return to battery 132, completing a circuit.

Atomizer housing 138 and neighboring components preferably define at least one atomizing liquid storage chamber, illustrated as storage chamber 140, and at least one atomizing chamber, illustrated as atomizing chamber 142. Liquid storage chamber 140 stores an atomizing liquid, to provide a consistent supply of the liquid to atomizing chamber 142 (at a restrained rate, through aperture 143), where the liquid enters into operative atomizing contact with atomizing element 154. Atomizing chamber 142 is a fluid conduit assembly designed to receive a supply of fluid, deliver it to atomizing element 154 in a reliable fashion, and integrate a certain portion of the atomized liquid into the fluid stream passing through the conduit by way of atomizing chamber 142. The path of breathable fluid flow through atomizer tank assembly 145 passes through the atomization element 154 and atomization chamber 142, where it then is introduced into the flow paths entering the exchangeable cartridge.

Liquid storage chamber is preferably constructed so as to prevent or inhibit unintended liquid leakage. It may also be constructed to permit disassembly or alteration to allow a user to refill the atomizing liquid reserve. Alternatively, it may be constructed to prevent disassembly or alteration by a user, and rather be intended to be a disposable device for one-time use. Desirable properties for materials of the chamber include chemical resistance, transparency, and structural strength and durability. Thus, one preferred material is polycarbonate. However, liquid storage chamber 140 may be constructed from any suitable material or materials chosen according to design specific parameters.

A liquid storage chamber and an atomizing chamber may alternatively combined into a single conduit element wherein a liquid retaining material (e.g. an absorbing or adsorbing material), such as a fibrous material, stores an atomizing liquid, releasing the atomizing liquid into contact with an atomizing element over a controlled area of the atomizing element for introduction into a breathable fluid stream at a restrained rate. Furthermore in contact with the liquid storage material would be an element for delivery of the stored liquid to a single atomizing mechanism or multiple atomizing mechanisms.

Atomizing element 154 may comprise electrically resistive wire, such as platinum, nichrome, or any other suitable metal alloy or material that may emit heat via electrical resistance, electrical induction, or any other comparable method for heat generation using a flow of electrons. Alternatively, atomizing element 154 may be a piezo-resistive nebulizer, pneumatic nebulizer, thin-film nebulizer, or any other mechanism whereby a said atomizing liquid and/or functional fluid may be aerosolized, nebulized, atomized, or otherwise introduced into a fluid air stream passing through the conduit channels of an inhalation device of the present invention. One or more of such atomizing mechanisms may be included within an atomizing chamber or elsewhere within the device.

Variations are also possible in the manner in which the atomizing mechanism interacts with the atomizing liquid, and the accompanying structures that cooperate with the atomizing mechanism. For example, when the atomizing mechanism comprises an atomizing element such as a hot wire coil, a cotton or fiberglass wick may be associated with the coil in order to draw atomizing liquid into the coil for atomization via capillary action. Also, in such embodiments, a rolled sheet of fiberglass or similar material may be packed into the chamber to assist in wicking atomizing liquid to the coil. In piezo-resistive or thin film nebulizers, often a pool of atomizing liquid is associated with the nebulizer in order to supply the liquid to it. In some cases, the nebulizer may not be in direct contact with the atomizing liquid, but may act upon the liquid by way of a pulse wave carrier fluid or media, which then interacts with a thin flexible film which then transfers the pulse wave energy to the atomizing liquid and in turn nebulizes the liquid. Any of these exemplary mechanistic approaches may be incorporated into devices according to the present invention, but by no means is the present invention limited to these designs. In summary, said atomizing chambers are not limited to any particular atomizing mechanism or any particular cooperating structures or materials required for the proper function thereof.

Advantageously, atomizing housing 138 may be configured to connect in line with exchangeable porous cartridge 144 containing a functional liquid. Thus, the atomizer housing 138 mates with cartridge housing cover 146 for receiving the cartridge, in which the cartridge is disposed in line with the fluid flow channel of the device, so as to expose the atomized fluid to the cartridge before the atomized fluid reaches a user. Preferably, a cartridge in housing cover 146 is retained in an easily accessible slot for tool-less removal and replacement. A connecting feature for connecting cartridge housing cover 146 to atomizer housing 138 may be a threaded connection as shown, a tight fitting sliding connection, lined with one or more O-rings, permitting the housings to be pushed together and pulled apart by a user, or any other suitable connection. Preferably the housing cover would act to cover and fix into place the exchangeable porous cartridge as well as define a space for attachment of a mouthpiece, if a mouthpiece is not integrated into the housing or housing cover.

In alternative embodiments of the exchangeable porous cartridge aspect of the invention, multiple cartridge pods, each pod comprising a packing material containing an absorbed or adsorbed functional liquid substantially as described with respect to the foregoing embodiments, may be incorporated into a single device. This may be done in many different ways, including, for example, by stacking multiple cartridges according to the foregoing embodiments, or by providing a single cartridge that houses multiple pods in a single casing.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. In an enhanced modular device for imparting a substance into a breathable fluid stream of an electronic cigarette, the improvement which comprises, in combination:
a fluid-permeable packing material containing a functional substance;
a housing configured to contain the packing material, the housing having a proximal end and a distal end in fluid communication with each other, wherein the proximal end is configured to be mated downstream of and in fluid communication with an atomizing apparatus, and the distal end is capable of drawing a fluid stream there through;
wherein when a fluid stream is drawn through the distal end of the housing, an atomized fluid from the atomizer passes through the packing material and the functional substance is imparted into the fluid stream; and
wherein the modular device includes a sealed disposable unit which precludes opening or releasing of any functional substance outside of exclusively with the atomizing apparatus.

2. The enhanced modular device of claim 1, wherein the packing material is situated within a casing that is at least partially fluid permeable at a proximal and distal end.

3. The enhanced modular device of claim 1, wherein the packing material and functional substance have been manufactured together such that no assembly is required from a user.

4. The enhanced modular device of claim 1, wherein the packing material is configured to be switched out by a user.

5. The enhanced modular device of claim 1, wherein the packing material is configured to have a functional substance added to it by a user.

6. The enhanced modular device of claim 1, wherein the housing further comprises one or more o-rings, gaskets or functional equivalents for creating a fluid-tight seal between the housing and an apparatus containing the atomizer.

7. The enhanced modular device of claim 1, wherein the distal end of the housing is connected to a mouthpiece.

8. The enhanced modular device of claim 1, wherein the functional substance is at least one of a liquid, a powder, and a flavoring.

9. The enhanced device of claim 1, wherein the functional substance comprises nicotine.

10. The enhanced device of claim 1, wherein the functional substance comprises at least one of propylene glycol, vegetable glycerin, nicotine, and flavoring.

11. A disposable e-liquid tank for use with an enhanced modular device for imparting a substance into a breathable fluid stream of an electronic cigarette, comprising:
a substantially cylindrical e-liquid reservoir, the reservoir comprising: an outer wall and an inner wall, the inner and outer walls connected at the distal end of the reservoir, thereby forming a substantially circular trough open to the proximal end; and a central bore defined by an inner surface of the inner wall, the bore extending axially through the reservoir and configured to fit around an atomizer;
an atomizer comprising a metal coil and a wicking material, the atomizer situated within the bore; and
a base coupled to the atomizer and situated over the open proximal end of the trough and forming a fluid-tight seal therewith, the base having a connector configured to connect to a battery unit for powering the atomizer.

12. The disposable e-liquid tank of claim 11, wherein the tank is pre-filled by a manufacturer and is configured to remain sealed throughout its use.

13. The disposable e-liquid tank of claim 11, wherein the tank is configured to release e-liquid in its atomized form but not in its liquid form.

14. The disposable e-liquid tank of claim 11, wherein the base further comprises a fill port configured to accept a needle for filling the tank.

15. The disposable e-liquid tank of claim 11, wherein the base further comprises a plug situated within the fill port to form a seal.

16. The disposable e-liquid tank of claim 11, wherein the disposable tank further comprises at least one of a flavoring, a medicament and a propylene glycol, vegetable glycerin and combinations thereof.

17. The disposable e-liquid tank of claim 12, wherein the inner wick controls the dosing of the liquid so there is a more consistent amount of liquid dispensed as the vapor flows through the center hole, since after each draw of vapor, the inner wick draws more liquid from the outer wick.

18. The disposable e-liquid tank of claim 11, wherein the top and bottom of the pod that hold the wick are cones that touch the inter wick at the hole through the center, whereby air flow does not dry the entire wick out while the pod is not in use.

19. The disposable e-liquid tank of claim 11, wherein the disposable tank further comprises at least one of a flavoring, a medicament and propylene glycol, vegetable glycerin and combinations thereof.

20. An enhanced modular device for imparting a substance into a breathable fluid stream of an electronic cigarette, the improvement which comprises, in combination:
an inner and an outer wick wherein the outer wick stores the liquid product in bulk, and the inner wick draws the liquid from the outer wick at a specific rate because dosing is controlled;
wherein the inner wick controls the dosing of the liquid so there is a more consistent amount of liquid dispensed as the vapor flows through the center hole, since after each draw of vapor, the inner wick draws more liquid from the outer wick; wherein the top and bottom of the pod that hold the wick are cones that touch the inter wick at the hole through the center, whereby air flow does not dry the entire wick out while the pod is not in use.
